(19) United States
(12) Patent Application Publication
(10) Pub. No.: US 2026/0188090 A1
(43) Pub. Date: Jul. 2, 2026

(54) ESCALATING VIBRATION OUTPUT BASED ON SLEEP STATE AND NOTIFICATION

(71) Applicant: Motorola Mobility LLC, Chicago, IL (US)

(72) Inventors: Daniel C. Chisu, Franklin Park, IL (US); Matthew Deheck, Vernon Hills, IL (US)

(73) Assignee: Motorola Mobility LLC, Chicago, IL (US)

(21) Appl. No.: 19/002,114

(22) Filed: Dec. 26, 2024

(51) Int. Cl. G08B 6/00; A61B 5/00; G08B 3/00

(52) U.S. Cl. CPC G08B 6/00; A61B 5/4806; G08B 3/00

(57) ABSTRACT

Techniques for escalating vibration output based on sleep state and notification are described and are implementable to enable a user to be made aware of a notification while the user is detected in a sleep state. For instance, a user may wear a wearable device that detects user attributes and generates sensor data that indicates that the user is in a sleep state. When a notification is received, such as on the user's mobile phone, an escalating vibration output can be triggered on the mobile phone and/or the wearable device.

### BACKGROUND

Today's mobile devices enable a variety of different usage scenarios, such as smartphones, laptops, tablets, wearable devices, and so forth. Further, mobile devices enable users to receive different types of notifications, such as voice calls, multimedia calls, alarms, calendar reminders, etc. However, if a user is sleeping when an important notification is received, the user may miss the important notification.

### BRIEF DESCRIPTION OF THE DRAWINGS

Aspects of escalating vibration output based on sleep state and notification are described with reference to the following Figures. The same numbers may be used throughout to reference similar features and components that are shown in the Figures. Further, identical numbers followed by different letters reference different instances of features and components described herein.

FIG. 1 illustrates an example environment in which aspects of escalating vibration output based on sleep state and notification can be implemented.

FIG. 2 illustrates a scenario in accordance with aspects of the present disclosure.

FIG. 3 illustrates a scenario in accordance with aspects of the present disclosure.

FIG. 4 illustrates a flow chart depicting an example method for escalating vibration output based on sleep state and notification in accordance with one or more implementations.

FIG. 5 illustrates a flow chart depicting an example method for escalating vibration output based on sleep state and notification in accordance with one or more implementations.

FIG. 6 illustrates various components of an example device in which aspects of escalating vibration output based on sleep state and notification can be implemented.

### DETAILED DESCRIPTION

Techniques for escalating vibration output based on sleep state and notification are described and are implementable to provide vibration (e.g., haptic) output for notifications when a user sleep state is detected. For example, consider a scenario in which a user is carrying a mobile phone and is wearing a wearable device, e.g., a smartwatch. Further, the user is resting and falls asleep, such while traveling on a train. While the user is asleep, the wearable device detects user attributes via sensors and generates sensor data, such as based on physiological and/or biometric attributes. Based on the user attributes, the sensor data can be used to detect that the user is asleep, e.g., a user sleep condition. Further, different sleep level states for sleep condition can be determined, such as a “light sleep” state, a “deep sleep” state, etc.

While the user is detected in the user sleep condition, the mobile phone and/or the wearable device detects a notification. The notification can take various forms, such as an incoming call, an alarm, a calendar event reminder, etc. In examples, the wearable device can notify the mobile phone of the user sleep condition prior to the notification being detected and/or concurrently with the notification. Accordingly, based on the user sleep condition and the notification, the mobile phone and/or the wearable device can output an escalating vibration output that increases in intensity over time. In examples, the escalating vibration output can be based on a detected sleep level state of the user, such as enable less intense vibration for a light sleep state and more intense vibration for a deep sleep state. The user may detect the escalating vibration output and awaken (e.g., transition to a wake state) to enable the user to consume (e.g., view, listen to) the notification.

Accordingly, techniques for escalating vibration output based on sleep state and notification can be implemented to enable users to be made aware of notifications while the users are in a sleep state.

While features and concepts of escalating vibration output based on sleep state and notification can be implemented in any number of environments and/or configurations, aspects of the described techniques are described in the context of the following example systems, devices, and methods. Further, the systems, devices, and methods described herein are interchangeable in various ways to provide for a wide variety of implementations and operational scenarios.

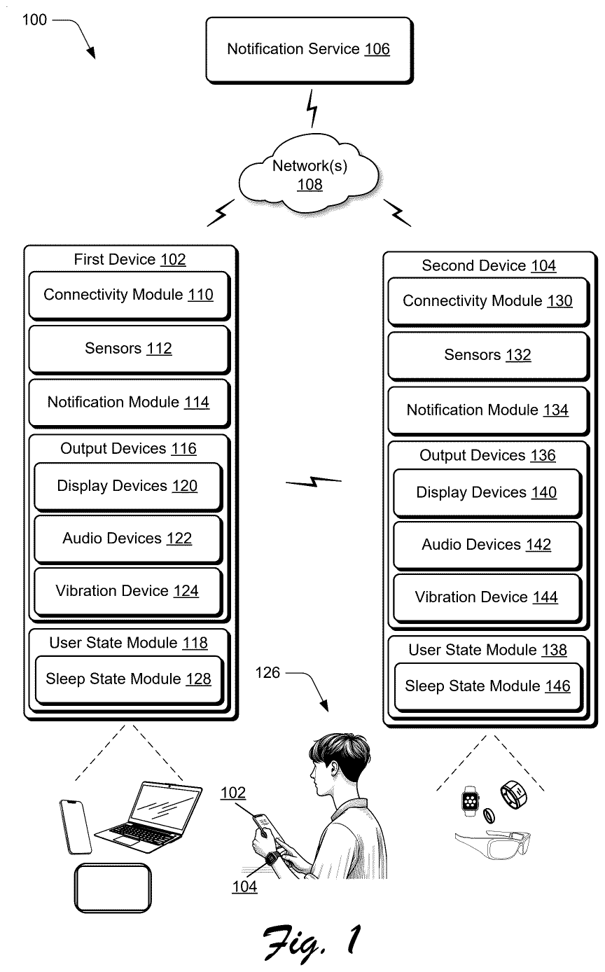

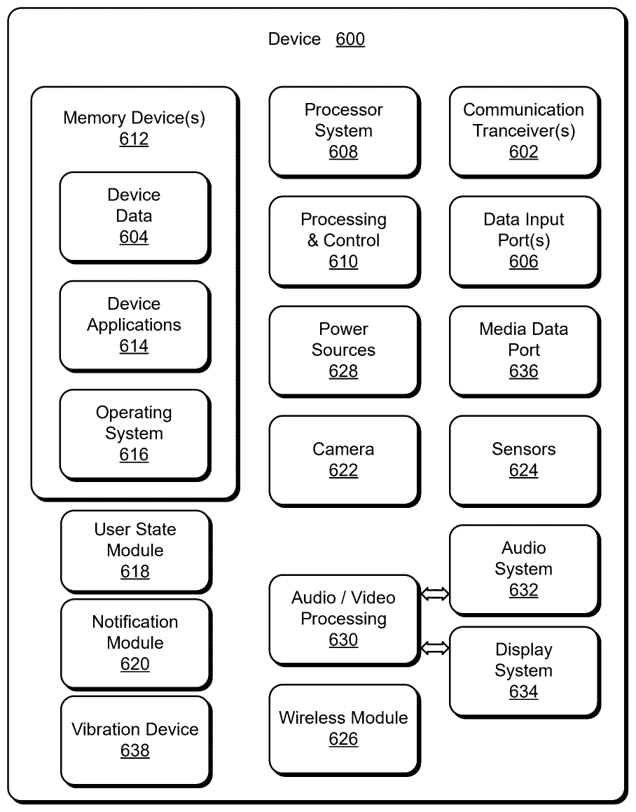

FIG. 1 illustrates an example environment 100 in which aspects of escalating vibration output based on sleep state and notification can be implemented. The environment 100 includes a first device 102, a second device 104, a notification service 106, and one or more network(s) 108. The first device 102 and the second device 104 represent devices that can be utilized by a user for different purposes, such as mobile devices that can be carried by a user to different locations. In at least one implementation, the first device 102 represents a mobile phone and the second device 104 represents a wearable device. These examples are not limiting, however, and the first device 102 and the second device 104 can be implemented in a variety of different ways. Example features and attributes of the first device 102 and the second device 104 are discussed below with reference to the device 600 of FIG. 6.

The first device 102 includes different functionality, including a connectivity module 110, sensors 112, a notification module 114, output devices 116, and a user state module 118. The connectivity module 110 represents functionality to enable wireless and wired connectivity of the first device 102, such as for wireless and/or wired connectivity to the network(s) 108. The connectivity module 110 may also enable direct device-to-device connectivity, such as for direct wireless connectivity between the first device 102 and the second device 104.

The sensors 112 are representative of functionality to detect various physical and/or logical phenomena in relation to the first device 102, such as motion, light, image detection and recognition, time and date, position, location, touch detection, sound (e.g., voice), temperature, biometric attributes, and so forth. Examples of the sensors 112 include hardware and/or logical sensors such as an accelerometer, a gyroscope, a camera, a microphone, a clock, biometric sensors, touch input sensors, position sensors, environmental sensors (e.g., for temperature, pressure, humidity, and so on), geographical location information sensors (e.g., Global Positioning System (GPS) functionality), and so forth. The sensors 112, however, can include a variety of other sensor types in accordance with the implementations discussed herein.

The notification module 114 represent functionality for managing notifications received at the first device 102, such as incoming notifications from other devices and network functionalities. Examples of notifications that the notification module 114 can manage include voice calls, text messages, multimedia calls, application notifications, system notifications, etc. The output devices 116 represent functionality for enabling different types of output via the first device 102, and include display devices 120, audio devices 122, and a vibration device 124. The display devices 120 represent functionality for visual output via the first device 102, such as for displaying graphics and/or other visual objects. The audio devices 122 represent functionality for audible output via the first device 102.

The vibration device 124 represents functionality for haptic output via the first device 102, such as vibrations and/or motions that are physically perceptible by a user of the first device 102. The vibration device 124, for example, can include motors and/or actuators that create physically perceptible vibration of one or more portions of the first device 102.

The user state module 118 is representative of functionality to monitor and detect different user states, such as user states associated with a user 126 of the first device 102. For instance, the sensors 112 can generate sensor data that can be utilized by the user state module 118 to monitor user states of the user 126. Further, the user state module 118 can receive data from other devices (e.g., the second device 104) to determine user state information. The user state module 118 includes a sleep state module 128 that can detect that the user 126 is in a sleep state, e.g., is sleeping. As further detailed throughout this disclosure, the notification module 114 can control different notifications based on detection of user sleep states.

Further to the environment 100, the second device 104 includes different functionality, including a connectivity module 130, sensors 132, a notification module 134, output devices 136, and a user state module 138. The connectivity module 130 represents functionality to enable wireless and wired connectivity of the second device 104, such as for wireless and/or wired connectivity to the network(s) 108. The connectivity module 130 may also enable direct device-to-device connectivity, such as for direct wireless connectivity between the second device 104 and the first device 102.

The sensors 132 are representative of functionality to detect various physical and/or logical phenomena in relation to the second device 104, such as motion, light, image detection and recognition, time and date, position, location, touch detection, sound (e.g., voice), temperature, biometric attributes, and so forth. Examples of the sensors 132 include hardware and/or logical sensors such as an accelerometer, a gyroscope, a camera, a microphone, a clock, biometric sensors, touch input sensors, position sensors, environmental sensors (e.g., for temperature, pressure, humidity, and so on), geographical location information sensors (e.g., Global Positioning System (GPS) functionality), and so forth. The sensors 132, however, can include a variety of other sensor types in accordance with the implementations discussed herein.

The notification module 134 represents functionality for managing notifications received at the second device 104, such as incoming notifications from other devices and network functionalities. Examples of notifications that the notification module 134 can manage include voice calls, text messages, multimedia calls, application notifications, system notifications, etc. The output devices 136 represent functionality for enabling different types of output via the second device 104, and include display devices 140, audio devices 142, and a vibration device 144. The display devices 140 represent functionality for visual output via the second device 104, such as for displaying graphics and/or other visual objects. The audio devices 142 represent functionality for audible output via the second device 104.

The vibration device 144 represents functionality for haptic output via the second device 104, such as vibrations and/or motions that are physically perceptible by a user of the second device 104. The vibration device 144, for example, can include motors and/or actuators that create physically perceptible vibration of one or more portions of the second device 104.

The user state module 138 is representative of functionality to monitor and detect different user states, such as user states associated with a user of the second device 104, e.g., the user 126. For instance, the sensors 132 can generate sensor data that can be utilized by the user state module 138 to monitor user states of the user 126. The user state module 138 includes a sleep state module 146 that can detect that the user 126 is in a sleep state, e.g., is sleeping. As further detailed throughout this disclosure, the notification module 134 can control different notifications based on detection of user sleep states.

The first device 102 and the second device 104 can be implemented in various ways and include various functionality, examples of which are discussed below with reference to the example device 600 of FIG. 6. Further, the network(s) 108 can represent a combination of wired and wireless networks via which the first device 102, the second device 104, and the notification service 106 can participate in various types of communication, such as wired and/or wireless data communication.

Having discussed an example environment in which the disclosed techniques can be performed, consider now some example scenarios and implementation details for implementing the disclosed techniques.

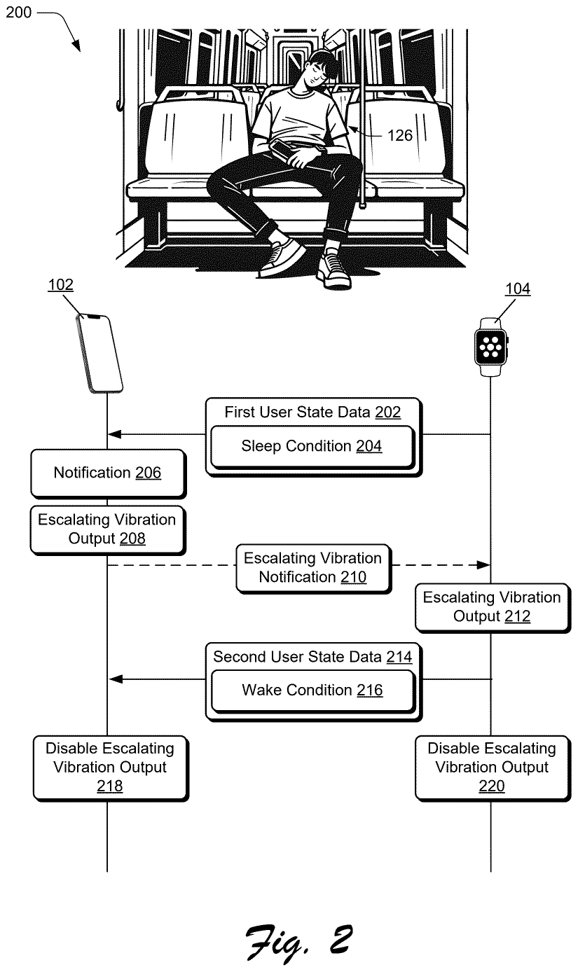

FIG. 2 illustrates a scenario 200 in accordance with aspects of the present disclosure. In the scenario 200, the user 126 is carrying the first device 102 and the second device 104 and is in a sleep state. The user 126, for example, is carrying the first device 102 (e.g., a mobile phone) and is wearing the second device 104, e.g., a wearable device such as a smartwatch. The second device 104 detects (e.g., based on biometric data from the sensors 132) that the user 126 is in a sleep state. The second device 104 transmits first user state data 202 indicating a sleep condition 204 to the first device 102. In at least one implementation, the first user state data 202 can indicate a sleep level state from multiple different sleep level states for the sleep condition. For instance, different sets of sensor data can indicate different sleep level states, such as light sleep, deep sleep, REM sleep, etc.

Further to the scenario 200, the first device 102 receives a notification 206, such as an incoming phone call, an alarm, a text message, a meeting request, etc. Accordingly, based on the sleep condition 204, the first device 102 outputs escalating vibration output 208. The escalating vibration output 208, for instance, is output as vibration output that increases in intensity over time, e.g., increasing frequency, velocity, acceleration, and/or displacement over time. In at least one implementation, the first device 102 can transmit an escalating vibration notification 210 to the second device 104, and the second device 104 can output escalating vibration output 212.

Further to the scenario 200, the second device 104 detects (e.g., based on biometric data from the sensors 112) that the user 126 transitions from the sleep state to an awake state. For instance, sensor data from the sensors 132 indicates that the user 126 transitions to an awake state, such as based on an increase in user movement, user speech, increase in heart rate, etc. Accordingly, the second device 104 transmits second user state data 214 indicating a wake condition 216 to the first device 102. Based on the wake condition 216, at 218 the first device 102 can disable escalating vibration output. For instance, the first device 102 can stop outputting vibration output and/or can deescalate vibration output over time. Further, and based on the wake condition 216, at 220 the second device 104 can disable escalating vibration output.

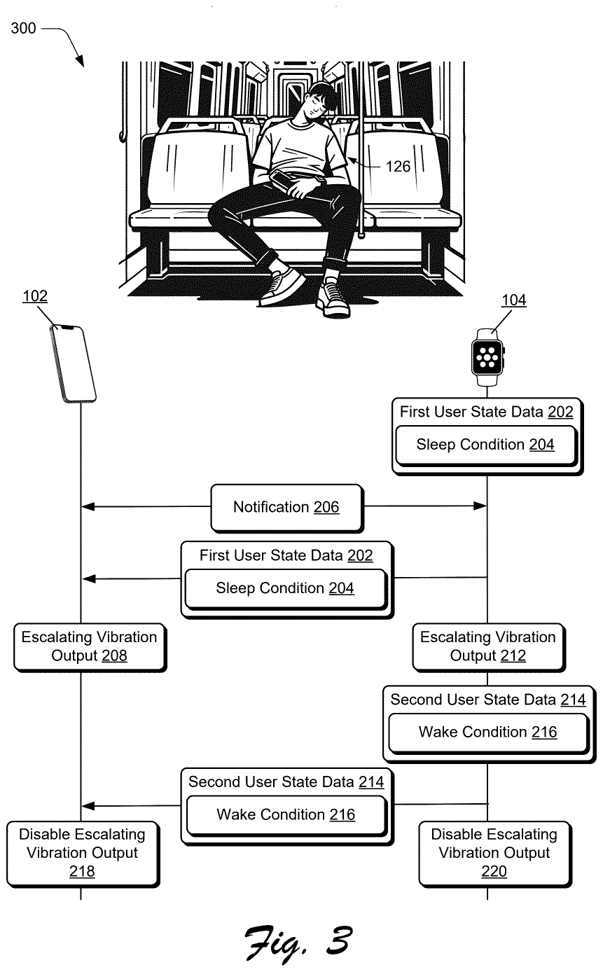

FIG. 3 illustrates a scenario 300 in accordance with aspects of the present disclosure. The scenario 300, for instance, represents an alternative or additional scenario to the scenario 200. In the scenario 300, the user 126 is carrying the first device 102 and the second device 104 and is in a sleep state. The user 126, for example, is carrying the first device 102 (e.g., a mobile phone) and is wearing the second device 104, e.g., a wearable device such as a smart watch. The second device 104 detects (e.g., based on biometric data from the sensors 132) that the user 126 is in a sleep state and generates the first user state data 202 indicating the sleep condition 204. Further, the second device 104 detects the notification 206, such as a notification received at the second device 104 and/or at the first device 102.

Based on detecting the notification 206, the second device 104 transmits the first user state data 202 indicating a sleep condition 204 to the first device 102. In at least one implementation, the first user state data 202 can indicate a sleep level state from multiple different sleep level states for the sleep condition. For instance, different sets of sensor data can indicate different sleep level states, such as light sleep, deep sleep, REM sleep, etc.

Further to the scenario 200, the first device 102 detects the notification 206 and based on the sleep condition 204, the first device 102 outputs the escalating vibration output 208. The escalating vibration output 208, for instance, is output as vibration output that increases in intensity over time, e.g., increasing frequency, velocity, acceleration, and/or displacement over time. In at least one implementation, the second device 104 can output escalating vibration output 212 based on the sleep condition 204 and the notification 206.

Further to the scenario 300, the second device 104 detects (e.g., based on biometric data from the sensors 112) that the user 126 transitions from the sleep state to an awake state. For instance, sensor data from the sensors 132 indicates that the user 126 transitions to an awake state, such as based on an increase in user movement, user speech, increase in heart rate, etc. Accordingly, the second device 104 transmits the second user state data 214 indicating the wake condition 216 to the first device 102. Based on the wake condition 216, at 218 the first device 102 can disable escalating vibration output. For instance, the first device 102 can stop outputting vibration output and/or can deescalate vibration output over time. Further, and based on the wake condition 216, at 220 the second device 104 can disable escalating vibration output.

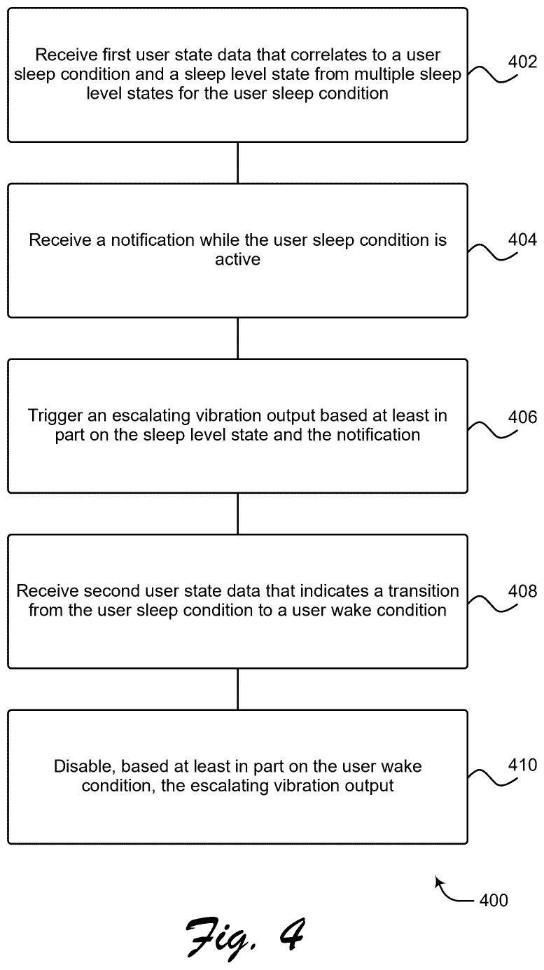

FIG. 4 illustrates a flow chart depicting an example method 400 for escalating vibration output based on sleep state and notification in accordance with one or more implementations. Operations of the method 400, for instance, may be performed in the context of the environment 100, such as by the first device 102 and/or the notification service 106.

At 402 first user state data is received that correlates to a user sleep condition and a sleep level state from multiple sleep level states for the user sleep condition. The first device 102, for instance, receives the first user state data from the second device 104. In an example, the first user state data can include (e.g., expressly identify) the user sleep condition and the sleep level state. Alternatively or additionally, the first user state data can include sensor data and the first device 102 can map the sensor data to sleep level data for the multiple sleep level states to determine the sleep level state from the multiple sleep level states.

At 404 a notification is received while the user sleep condition is active. The first device 102, for example, receives (e.g., detects) a notification while the user sleep condition is in effect, such as an incoming call, an alarm, a calendar reminder, a message, etc. At 406 an escalating vibration output is triggered based at least in part on the sleep level state and the notification. In examples, each sleep level state is associated with a different respective vibration escalation profile for the escalating vibration output. Further, each vibration escalation profile can include a different increase in vibration intensity over time. For instance, a “light sleep” sleep level state can correspond to a lower intensity vibration escalation profile and a “deep sleep” sleep level state can correspond to a higher intensity vibration escalation profile. Further, the first device 102 can trigger an escalating audio output in conjunction with the escalating vibration output, and based at least in part on the sleep level state and the notification. In an example, the first device 102 can transmit, to the second device 104, an instruction to implement the escalating vibration output while the notification is pending.

At 408 second user state data is received that indicates a transition from the user sleep condition to a user wake condition. The first device 102, for instance, receives the second user state data from the second device 104. In an example, the second user state data can include (e.g., expressly identify) the user wake condition. Alternatively or additionally, the second user state data can include sensor data and the first device 102 can map the sensor data to user state data that correlates to a user wake condition. At 410, based at least in part on the user wake condition, the escalating vibration output is disabled. The first device 102, for example, can immediately terminate the escalating vibration output or may gradually decrease the vibration output, e.g., via a gradually deescalating vibration output over time.

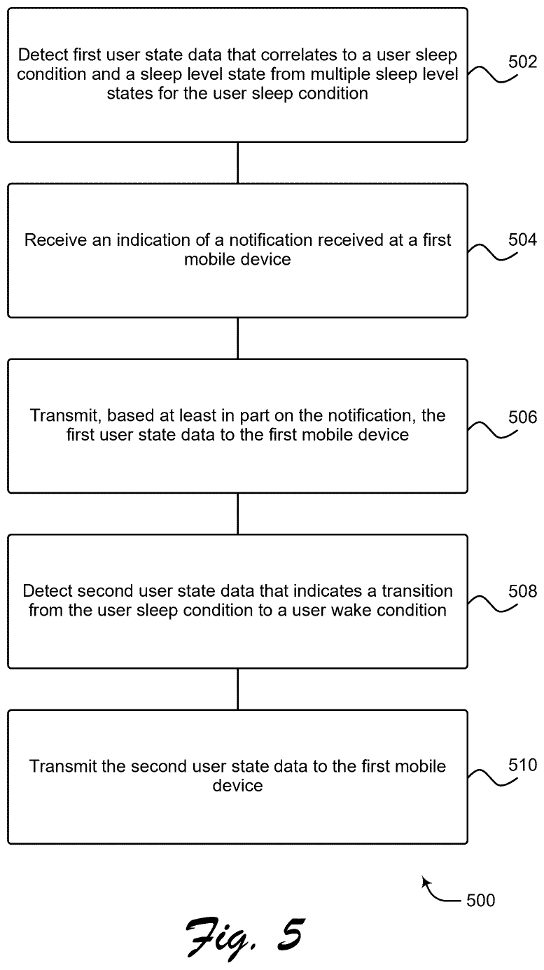

FIG. 5 illustrates a flow chart depicting an example method 500 for escalating vibration output based on sleep state and notification in accordance with one or more implementations. Operations of the method 500, for instance, may be performed in the context of the environment 100, such as by the second device 104 and/or the notification service 106. At 502, first user state data is detected that correlates to a user sleep condition and a sleep level state from multiple sleep level states for the user sleep condition. The second device 104, for example, collects sensor data based on user attributes, such as physiological and/or biometric user attributes. In examples, the first user state data may include the collected sensor data. Alternatively or additionally, the second device 104 may map the sensor data to the sleep level state from the multiple sleep level states, and include the sleep condition and the sleep level state in the first user state data.

At 504, an indication is received of a notification received at a first mobile device. The second device 104, for example, detects that a notification is received at the first device 102. At 506, based at least in part on the notification, the first user state data is transmitted to the first mobile device. The second device 104, for example, transmits the first user state data to the first device 102. In examples, the second device 104 can transmit the first user state data to the second device 104 based at least in part on a query from the first device 102 for the first user state data. In examples, the second device 104 can receive, from the first device 102, an instruction to implement an escalating vibration output, and the second device 104 can output the escalating vibration output while the notification is pending.

At 508, second user state data is detected that indicates a transition from the user sleep condition to a user wake condition. At 510, the second user state data is transmitted to the first mobile device. The second device 104, for example, can transmit the second user state data to the first device 102. In an example, the second user state data can include (e.g., expressly identify) the user wake condition. Alternatively or additionally, the second user state data can include sensor data that correlates to the user wake condition. In examples, the second device 104 can disable, based at least in part on the user wake condition, an escalating vibration output for the notification.

The example methods described above may be performed in various ways, such as for implementing different aspects of the systems and scenarios described herein. Generally, any services, components, modules, methods, and/or operations described herein can be implemented using software, firmware, hardware (e.g., fixed logic circuitry), manual processing, or any combination thereof. Some operations of the example methods may be described in the general context of executable instructions stored on computer-readable storage memory that is local and/or remote to a computer processing system, and implementations can include software applications, programs, functions, and the like. Alternatively or in addition, any of the functionality described herein can be performed, at least in part, by one or more hardware logic components, such as, and without limitation, Field-programmable Gate Arrays (FPGAs), Application-specific Integrated Circuits (ASICs), Application-specific Standard Products (ASSPs), System-on-a-chip systems (SoCs), Complex Programmable Logic Devices (CPLDs), and the like. The order in which the methods are described is not intended to be construed as a limitation, and any number or combination of the described method operations can be performed in any order to perform a method, or an alternate method.

FIG. 6 illustrates various components of an example device 600 in which aspects of escalating vibration output based on sleep state and notification can be implemented. The example device 600 can be implemented as any of the devices described with reference to the previous FIGS. 1-5, such as any type of mobile device, mobile phone, mobile device, wearable device, tablet, computing, communication, entertainment, gaming, media playback, and/or other type of electronic device. For example, the first device first device 102, the second device 104, and/or the notification service 106 as shown and described with reference to FIGS. 1-5 may be implemented as the example device 600.

The device 600 includes communication transceivers 602 that enable wired and/or wireless communication of device data 604 with other devices. The device data 604 can include one or more of device identifying data, device location data, wireless connectivity data, and wireless protocol data. Additionally, the device data 604 can include any type of audio, video, and/or image data. Example communication transceivers 602 include wireless personal area network (WPAN) radios compliant with various IEEE 802.15 (Bluetooth™) standards, wireless local area network (WLAN) radios compliant with any of the various IEEE 802.10 (Wi-Fi™) standards, wireless wide area network (WWAN) radios for cellular phone communication, wireless metropolitan area network (WMAN) radios compliant with various IEEE 802.16 (WiMAX™) standards, and wired local area network (LAN) Ethernet transceivers for network data communication.

The device 600 may also include one or more data input ports 606 via which any type of data, media content, and/or inputs can be received, such as user-selectable inputs to the device, messages, music, television content, recorded content, and any other type of audio, video, and/or image data received from any content and/or data source. The data input ports may include USB ports, coaxial cable ports, and other serial or parallel connectors (including internal connectors) for flash memory, DVDs, CDs, and the like. These data input ports may be used to couple the device to any type of components, peripherals, or accessories such as microphones and/or cameras.

The device 600 includes a processing system 608 of one or more processors (e.g., any of microprocessors, controllers, and the like) and/or a processor and memory system implemented as a system-on-chip (SoC) that processes computer-executable instructions. The processor system may be implemented at least partially in hardware, which can include components of an integrated circuit or on-chip system, an application-specific integrated circuit (ASIC), a field-programmable gate array (FPGA), a complex programmable logic device (CPLD), and other implementations in silicon and/or other hardware. Alternatively or in addition, the device can be implemented with any one or combination of software, hardware, firmware, or fixed logic circuitry that is implemented in connection with processing and control circuits, which are generally identified at 610. The device 600 may further include any type of a system bus or other data and command transfer system that couples the various components within the device. A system bus can include any one or combination of different bus structures and architectures, as well as control and data lines.

The device 600 also includes computer-readable storage memory 612 (e.g., memory devices) that enable data storage, such as data storage devices that can be accessed by a computing device, and that provide persistent storage of data and executable instructions (e.g., software applications, programs, functions, and the like). Examples of the computer-readable storage memory 612 include volatile memory and non-volatile memory, fixed and removable media devices, and any suitable memory device or electronic data storage that maintains data for computing device access. The computer-readable storage memory can include various implementations of random access memory (RAM), read-only memory (ROM), flash memory, and other types of storage media in various memory device configurations. The device 600 may also include a mass storage media device.

The computer-readable storage memory 612 provides data storage mechanisms to store the device data 604, other types of information and/or data, and various device applications 614 (e.g., software applications). For example, an operating system 616 can be maintained as software instructions with a memory device and executed by the processing system 608. The device applications may also include a device manager, such as any form of a control application, software application, signal-processing and control module, code that is native to a particular device, a hardware abstraction layer for a particular device, and so on. Computer-readable storage memory 612 represents media and/or devices that enable persistent and/or non-transitory storage of information in contrast to mere signal transmission, carrier waves, or signals per se. Computer-readable storage memory 612 do not include signals per se or transitory signals.

In this example, the device 600 includes a user state module 618 and notification module 620 that can implement aspects of escalating vibration output based on sleep state and notification and may be implemented with hardware components and/or in software. For example, the user state module 618 can be implemented as the user state module 118 and/or the user state module 138. Further, the notification module 620 can be implemented as the notification module 114 and/or the notification module 134. In implementations, the user state module 618 and/or the notification module 620 may include independent processing, memory, and logic components as a computing and/or electronic device integrated with the device 600.

In this example, the example device 600 also includes a camera 622 and sensors 624. The sensors 624 can be implemented in various ways and are representative of functionality to detect various physical and/or logical phenomena in relation to the device 600, such as motion, light, image detection and recognition, time and date, position, location, touch detection, sound, temperature, and so forth. Examples of the sensors 624 include hardware and/or logical sensors such as an accelerometer, a gyroscope, a camera, a microphone, a clock, biometric sensors, touch input sensors, position sensors, environmental sensors (e.g., for temperature, pressure, humidity, and so on), geographical location information sensors (e.g., Global Positioning System (GPS) functionality), and so forth.

The device 600 also includes a wireless module 626, which is representative of functionality to perform various wireless communication tasks. The device 600 can also include one or more power sources 628, such as when the device is implemented as a mobile device. The power sources 628 may include a charging and/or power system, and can be implemented as a flexible strip battery, a rechargeable battery, a charged super-capacitor, and/or any other type of active or passive power source.

The device 600 also includes an audio and/or video processing system 630 that generates audio data for an audio system 632 and/or generates display data for a display system 634. The audio system and/or the display system may include any devices that process, display, and/or otherwise render audio, video, display, and/or image data. Display data and audio signals can be communicated to an audio component and/or to a display component via an RF (radio frequency) link, S-video link, HDMI (high-definition multimedia interface), composite video link, component video link, DVI (digital video interface), analog audio connection, or other similar communication link, such as media data port 636. In implementations, the audio system and/or the display system are integrated components of the example device. Alternatively, the audio system and/or the display system are external, peripheral components to the example device. The device 600 also includes a vibration device 638 that can be implemented to provide vibration output, such as haptic output for the device 600. In examples, the vibration device 638 can be implemented as the vibration device 124 and or the vibration device 144.

Although implementations of escalating vibration output based on sleep state and notification have been described in language specific to features and/or methods, the subject of the appended claims is not necessarily limited to the specific features or methods described. Rather, the features and methods are disclosed as example implementations, and other equivalent features and methods are intended to be within the scope of the appended claims. Further, various different examples are described and it is to be appreciated that each described example can be implemented independently or in connection with one or more other described examples. Additional aspects of the techniques, features, and/or methods discussed herein relate to one or more of the following:

In some aspects, the techniques described herein relate to a first mobile device including: at least one memory; and at least one processor coupled with the at least one memory and configured to cause the first mobile device to: receive first user state data that correlates to a user sleep condition and a sleep level state from multiple sleep level states for the user sleep condition; receive a notification while the user sleep condition is active; and trigger an escalating vibration output based at least in part on the sleep level state and the notification.

In some aspects, the techniques described herein relate to a first mobile device, wherein the at least one processor is configured to cause the first mobile device to receive the first user state data from a second mobile device.

In some aspects, the techniques described herein relate to a first mobile device, wherein the at least one processor is configured to cause the first mobile device to transmit, to the second mobile device, a query for the first user state data.

In some aspects, the techniques described herein relate to a first mobile device, wherein the at least one processor is configured to cause the first mobile device to transmit, to the second mobile device, an instruction to implement the escalating vibration output while the notification is pending.

In some aspects, the techniques described herein relate to a first mobile device, wherein each of the multiple sleep level states is based on a different respective set of sleep data indicators.

In some aspects, the techniques described herein relate to a first mobile device, wherein each set of sleep data indicators includes a respective set of sensor data.

In some aspects, the techniques described herein relate to a first mobile device, wherein the at least one processor is configured to cause the first mobile device to trigger an escalating audio output in conjunction with the escalating vibration output, and based at least in part on the sleep level state and the notification.

In some aspects, the techniques described herein relate to a first mobile device, wherein each sleep level state is associated with a different respective vibration escalation profile for the escalating vibration output.

In some aspects, the techniques described herein relate to a first mobile device, wherein each vibration escalation profile includes a different increase in vibration intensity over time.

In some aspects, the techniques described herein relate to a first mobile device, wherein the first user state data includes sensor data received from a second mobile device, and wherein the at least one processor is configured to cause the first mobile device to: map the sensor data to sleep level data for the multiple sleep level states to determine the sleep level state from the multiple sleep level states.

In some aspects, the techniques described herein relate to a first mobile device, wherein the at least one processor is configured to cause the first mobile device to: receive second user state data that indicates a transition from the user sleep condition to a user wake condition; and disable, based at least in part on the user wake condition, the escalating vibration output.

In some aspects, the techniques described herein relate to a second mobile device including: at least one memory; and at least one processor coupled to the at least one memory and configured to cause the second mobile device to: detect first user state data that correlates to a user sleep condition and a sleep level state from multiple sleep level states for the user sleep condition; receive an indication of a notification received at a first mobile device; and transmit, based at least in part on the notification, the first user state data to the first mobile device.

In some aspects, the techniques described herein relate to a second mobile device, wherein the at least one processor is configured to cause the second mobile device to transmit the first user state data to the second mobile device based at least in part on a query from the first mobile device for the first user state data.

In some aspects, the techniques described herein relate to a second mobile device, wherein the at least one processor is configured to cause the second mobile device to: receive, from the first mobile device, an instruction to implement an escalating vibration output; and output the escalating vibration output while the notification is pending.

In some aspects, the techniques described herein relate to a second mobile device, wherein each sleep level state is associated with a different respective vibration escalation profile for the escalating vibration output.

In some aspects, the techniques described herein relate to a second mobile device, wherein the at least one processor is configured to cause the second mobile device to: detect sensor data from one or more sensors of the second mobile device; and map the sensor data to sleep level data for the multiple sleep level states to determine the sleep level state from the multiple sleep level states.

In some aspects, the techniques described herein relate to a second mobile device, wherein each sleep level state of the multiple sleep level states is associated with a different respective set of sensor data.

In some aspects, the techniques described herein relate to a second mobile device, wherein the at least one processor is configured to cause the second mobile device to: detect second user state data that indicates a transition from the user sleep condition to a user wake condition; and transmit the second user state data to the first mobile device.

In some aspects, the techniques described herein relate to a second mobile device, wherein the at least one processor is configured to cause the second mobile device to: disable, based at least in part on the user wake condition, an escalating vibration output for the notification.

In some aspects, the techniques described herein relate to a method performed by a mobile device, the method including: receiving first user state data that correlates to a user sleep condition and a sleep level state from multiple sleep level states for the user sleep condition; receiving a notification while the user sleep condition is active; and triggering an escalating vibration output based at least in part on the sleep level state and the notification.

1. A first mobile device comprising:
at least one memory; and
at least one processor coupled with the at least one memory and configured to cause the first mobile device to:
receive first user state data that correlates to a user sleep condition and a sleep level state from multiple sleep level states for the user sleep condition;
receive a notification while the user sleep condition is active; and
trigger an escalating vibration output based at least in part on the sleep level state and the notification.

2. The first mobile device of claim 1, wherein the at least one processor is configured to cause the first mobile device to receive the first user state data from a second mobile device.

3. The first mobile device of claim 2, wherein the at least one processor is configured to cause the first mobile device to transmit, to the second mobile device, a query for the first user state data.

4. The first mobile device of claim 2, wherein the at least one processor is configured to cause the first mobile device to transmit, to the second mobile device, an instruction to implement the escalating vibration output while the notification is pending.

5. The first mobile device of claim 1, wherein each of the multiple sleep level states is based on a different respective set of sleep data indicators.

6. The first mobile device of claim 5, wherein each set of sleep data indicators comprises a respective set of sensor data.

7. The first mobile device of claim 1, wherein the at least one processor is configured to cause the first mobile device to trigger an escalating audio output in conjunction with the escalating vibration output, and based at least in part on the sleep level state and the notification.

8. The first mobile device of claim 1, wherein each sleep level state is associated with a different respective vibration escalation profile for the escalating vibration output.

9. The first mobile device of claim 8, wherein each vibration escalation profile comprises a different increase in vibration intensity over time.

10. The first mobile device of claim 1, wherein the first user state data comprises sensor data received from a second mobile device, and wherein the at least one processor is configured to cause the first mobile device to:
map the sensor data to sleep level data for the multiple sleep level states to determine the sleep level state from the multiple sleep level states.

11. The first mobile device of claim 1, wherein the at least one processor is configured to cause the first mobile device to:
receive second user state data that indicates a transition from the user sleep condition to a user wake condition; and
disable, based at least in part on the user wake condition, the escalating vibration output.

12. A second mobile device comprising:
at least one memory; and
at least one processor coupled to the at least one memory and configured to cause the second mobile device to:
detect first user state data that correlates to a user sleep condition and a sleep level state from multiple sleep level states for the user sleep condition;
receive an indication of a notification received at a first mobile device; and
transmit, based at least in part on the notification, the first user state data to the first mobile device.

13. The second mobile device of claim 12, wherein the at least one processor is configured to cause the second mobile device to transmit the first user state data to the second mobile device based at least in part on a query from the first mobile device for the first user state data.

14. The second mobile device of claim 12, wherein the at least one processor is configured to cause the second mobile device to:
receive, from the first mobile device, an instruction to implement an escalating vibration output; and
output the escalating vibration output while the notification is pending.

15. The second mobile device of claim 14, wherein each sleep level state is associated with a different respective vibration escalation profile for the escalating vibration output.

16. The second mobile device of claim 12, wherein the at least one processor is configured to cause the second mobile device to:
detect sensor data from one or more sensors of the second mobile device; and
map the sensor data to sleep level data for the multiple sleep level states to determine the sleep level state from the multiple sleep level states.

17. The second mobile device of claim 16, wherein each sleep level state of the multiple sleep level states is associated with a different respective set of sensor data.

18. The second mobile device of claim 12, wherein the at least one processor is configured to cause the second mobile device to:
detect second user state data that indicates a transition from the user sleep condition to a user wake condition; and
transmit the second user state data to the first mobile device.

19. The second mobile device of claim 18, wherein the at least one processor is configured to cause the second mobile device to:
disable, based at least in part on the user wake condition, an escalating vibration output for the notification.

20. A method performed by a mobile device, the method comprising:
receiving first user state data that correlates to a user sleep condition and a sleep level state from multiple sleep level states for the user sleep condition;
receiving a notification while the user sleep condition is active; and
triggering an escalating vibration output based at least in part on the sleep level state and the notification.
